(19) United States
(12) Patent Application Publication
(10) Pub. No.: US 2003/0119834 A1
(43) Pub. Date: Jun. 26, 2003

(54) Compositions and methods of treatment of cancer

(76) Inventor: Cynthia C. Bamdad, Newton, MA (US)

Correspondence Address: Timothy J. Oyer, Wolf, Greenfield & Sacks, P.C., 600 Atlantic Avenue, Boston, MA, 02210, US

(21) Appl. No.: 10/236,863

(22) Filed: Sep. 5, 2002

Related U.S. Application Data
(60) Provisional application No. 60/317,314, filed on Sep. 5, 2001.

(51) Int. Cl. A61K 31/53; A61K 31/517

(52) U.S. Cl. 514/241; 514/252.02; 514/255.05; 514/266.21; 514/266.3

(57) ABSTRACT

The present invention generally relates to compositions useful in the treatment or prevention of cancer, in some cases by limiting or preventing angiogenesis. Other compositions useful for the treatment or prevention of cancer or angiogenesis include homologs, analogs, derivatives, enantiomers or functionally equivalent compositions of the present invention. The present compositions can be packaged in kits. The present invention also relates to the use of compositions useful for the treatment of patients susceptible to or exhibiting symptoms characteristic of cancer, for example, patients with solid tumors.

### RELATED APPLICATIONS

This non-provisional application claims the benefit under Title 35 U.S.C. §19(e) of co-pending U.S. provisional patent application serial No. 60/317,314, filed Sep. 5, 2001, entitled “Compositions and Methods of Treatment of Cancer,” by C. Bamdad et al., incorporated herein by reference.

### FIELD OF THE INVENTION

This invention generally relates to compositions and methods for cancer treatment, and in particular to treatments of cancer using angiogenesis inhibitors.

### DESCRIPTION OF THE RELATED ART

Angiogenesis is the name given to the in vivo process of new blood vessel formation. Angiogenesis inhibitors are a class of molecules that can interrupt this process of vascularization. It is believed that many forms of cancer can be effectively treated by reducing or eliminating the supply of blood to a tumor. Tumors cannot grow beyond a diameter of about 5 to 7 mm without developing their own system of blood vessels. Vascularization or angiogenesis thus enables a tumor to have ready access to a source of nutrients, which can allow it to grow and potentially metastasize. Because angiogenesis does not typically occur in adults unless associated with wound healing, it has been suggested that angiogenesis inhibitors may be effective treatments against cancer while minimizing many negative side effects.

It has been recently discovered that patients with large primary tumors produce two proteins, named angiostatin and endostatin. After surgical removal of the primary tumor, an event which can often triggers aggressive metastasis, it was found that some patients cease production of those proteins. Angiostatin and endostatin have since been shown to inhibit angiogenesis. Evidence has been presented that has shown administration of these proteins to animals with cancerous tumors can result in the inhibition of the growth of the tumors, possibly by removing the blood supply to the tumors. One theory put forth to explain these observations is that the primary tumor, after vascularization, signals the production of these proteins to block new blood vessel formation in the rest of the body. Thus, the primary tumor “reserves” nutrients for itself, which may cause distant metastases lay dormant. The removal of the primary tumor causes a decrease in the production of angiostatin and endostatin, which may enable distant metastases to vascularize, grow, or metastasize.

However, one potential drawback of using angiostatin and endostatin as cancer therapeutics is that they may be hard to administer, easily degraded by the body, or expensive to produce. For these reasons, it would be advantageous to have a rapid method for identifying new compounds (e.g., synthetic compounds), that can act to inhibit angiogenesis.

It has heretofore been difficult to identify new angiogenesis inhibitors, as the biological process of vascularization is not well understood. There have been few available defined molecular targets for use in drug screening. Additionally, many assays used to identify new angiogenesis inhibitors are functional, cell-based assays and cannot easily achieve high throughput rates.

The cell surface receptor, alpha-V-beta-3 (αᵥβ₃), has been implicated in promoting metastasis and angiogenesis (Li, X., Regezi, J., Ross, F. P., Blystone, S., Llic, D., Leong, S. P., and Ramos, D. M., “Integrin αᵥβ₃ mediates K1735 murine melanoma cell motility in vivo and in vitro,” 2001, J. Cell. Sci., Vol. 114 (14):2665-2672). It has been suggested that this receptor mediates angiogenesis through an interaction with a cell adhesion molecule, vitronectin (Hynes, R. O., 1987, Cell, Vol. 48:549-554). Specifically, it is the GRGDS motif derived from vitronectin that the alpha-V-beta-3 receptor is believed to bind to (Standker, L., Enger, A., Schalz-Knappe, P., Wohn, K., Matthias, G., Raida, M., Forssmann, W., and Preissner, K. T., 1996, Eur. J. Biochem., Vol. 241:557-554). Peptides that contain tandem repeats of GRGDS motifs may inhibit the binding of vitronectin to the alpha-V-beta-3 receptor, which has been shown to promote angiogenesis.

### SUMMARY OF THE INVENTION

The present invention involves, in one aspect, methods for treating patients susceptible or exhibiting symptoms of cancer, and in particular, solid tumors. The methods may involve, for example, the administration of angiogenesis inhibitors.

The subject matter of this application involves, in some cases, interrelated products, alternative solutions to a particular problem, and/or a plurality of different uses of a single system or article.

In one aspect, the invention provides a pharmaceutical preparation comprising a composition and a pharmaceutically acceptable carrier. In one embodiment, the composition can be any one of compositions 1-31. In another embodiment, the composition comprises homologs, analogs, derivatives, enantiomers and functionally equivalent compositions thereof of compositions 1-31.

In all structures herein, atom locations, if unlabeled, are carbon with appropriate hydrogen(s).

In one embodiment of the invention, the composition includes a structure:

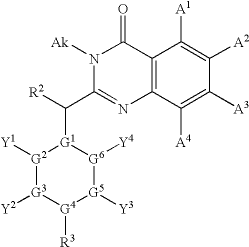

where A¹, A², A³, and A⁴ are each independently selected from the group consisting of H and a halogen, Y¹, Y², Y³, Y⁴, R² and R³ each independently comprise an atom, G¹, G², G³, G⁴, G⁵, and G⁶ each independently comprise an atom able to form at least three covalent bonds, and Ak comprises an alkyl.

In another embodiment of the invention, the composition includes a structure:

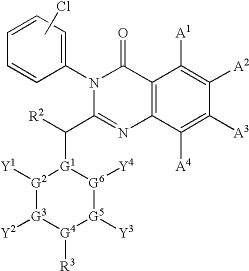

where A¹, A², A³, and A⁴ are each independently selected from the group consisting of H and a halogen, Y¹, Y², Y³, Y⁴, R² and R³ each independently comprise an atom, and G¹, G², G³, G⁴, G⁵, and G⁶ each independently comprise an atom able to form at least three covalent bonds. —Cl, as depicted, can be bound to any of the available verticies of the ring from which it emanates. This interpretation applies to other, similarly-depicted structures herein.

In another embodiment of the invention, the composition includes a structure:

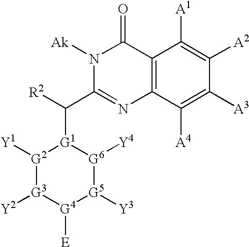

where A¹, A², A³, and A⁴ are each independently selected from the group consisting of H and a halogen, Y¹, Y², Y³, Y⁴, R¹ and R² each independently comprise an atom, G¹, G², G³, G⁴, G⁵, and G⁶ each independently comprise an atom able to form at least three covalent bonds, and E comprises a sulfur atom.

In another embodiment of the invention, the composition includes a structure:

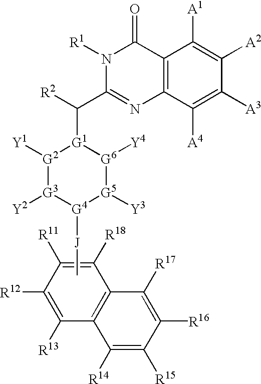

where A¹, A², A³, and A⁴ are each independently selected from the group consisting of H and a halogen, Y¹, Y², Y³, Y⁴, R¹, R², R¹¹, R¹², R¹³, R¹⁴, R¹⁵, R¹⁶, R¹⁷, and R¹⁸ each independently comprise an atom, G¹, G², G³, G⁴, G⁵, and G⁶ each independently comprise an atom able to form at least three covalent bonds, and J comprises a chemical bond or an atom.

In another embodiment of the invention, the composition includes a structure:

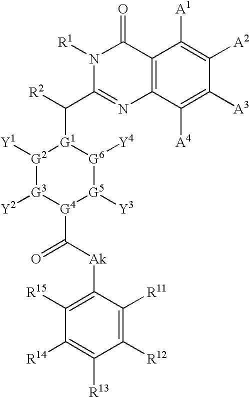

where A¹, A², A³, and A⁴ are each independently selected from the group consisting of H and a halogen, Y¹, Y², Y³, Y⁴, R¹, R², R¹¹, R¹², R¹³, R¹⁴, and R¹⁵ each independently comprise an atom, and G¹, G², G³, G⁴, G⁵, and G⁶ each independently comprise an atom able to form at least three covalent bonds.

In another embodiment of the invention, the composition includes a structure:

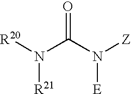

where R²⁰ and R²¹ each independently comprise an atom, E comprises at least 2 cyclic groups, and Z comprises at least two fused cyclic structures; in combination with a pharmaceutically acceptable carrier.

In another embodiment of the invention, the composition includes a structure:

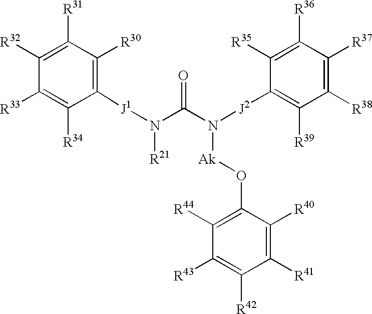

wherein Ak comprises a non-heteroatom alkyl group or is free of non-terminal heteroatoms, R²¹, R³⁰, R³¹, R³², R³³, R³⁴, R³⁵, R³⁶, R³⁷, R³⁸, R³⁹, R⁴⁰, R⁴¹, R⁴², R⁴³, and R⁴⁴ each independently comprise an atom, and J¹ and J² each independently comprise a chemical bond or an atom; in combination with a pharmaceutically acceptable carrier.

In another embodiment of the invention, the composition includes a structure:

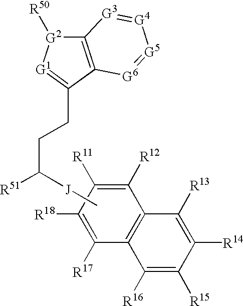

where R¹¹, R¹², R¹³, R¹⁴, R¹⁵, R¹⁶, R¹⁷, R¹⁸, R⁵⁰, and R⁵¹ each independently comprise an atom, G¹, G², G³, G⁴, and G⁵ each independently comprise an atom able to form at least three covalent bonds, and J comprises a chemical bond or an atom; in combination with a pharmaceutically acceptable carrier.

In another embodiment of the invention, the composition includes a structure:

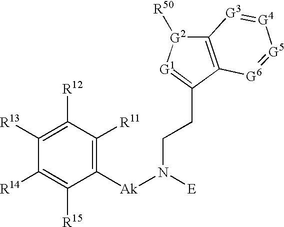

where R¹¹, R¹², R¹³, R¹⁴, R¹⁵, and R⁵⁰ each independently comprise an atom, G¹, G², G³, G⁴, and G⁵ each independently comprise an atom able to form at least three covalent bonds, Ak comprises an alkyl, and E comprises a sulfur atom; in combination with a pharmaceutically acceptable carrier.

In another embodiment of the invention, the composition includes a structure:

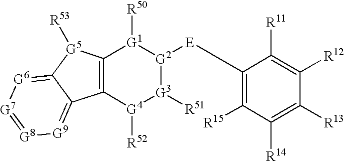

where R¹¹, R¹², R¹³, R¹⁴, R¹⁵, R⁵⁰, R⁵¹, R⁵², and R⁵³ each independently comprise an atom, G¹, G², G³, G⁴, G⁵, G⁶, G⁷, G⁸, and G⁹ each independently comprise an atom able to form at least three covalent bonds, and E comprises a sulfur atom; in combination with a pharmaceutically acceptable carrier.

In one aspect, the invention comprises a method. In one embodiment, the method is defined, at least in part, by the step of treating a human patient susceptible to or exhibiting a solid tumor, by administering to the patient a therapeutically effective amount of a composition that inhibits the tumor by inhibiting angiogenesis, comprising:

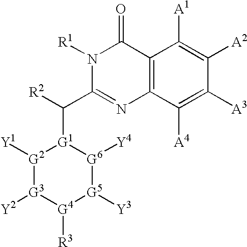

wherein A¹, A², A³, and A⁴ are each independently selected from the group consisting of H and a halogen, Y¹, Y², Y³, Y⁴, R¹, R² and R³ each independently comprise an atom, G¹, G², G³, G⁴, G⁵, and G⁶ each independently comprise an atom able to form at least three covalent bonds, and the patient is not otherwise indicated for treatment with the composition.

The invention includes methods of treatment of selected groups of patients. It is to be understood that all compositions described herein are useful for each described method. In one set of embodiments, the patient is susceptible to, but does not exhibit symptoms of, the disease of cancer (e.g. solid tumors). In another set of embodiments, the patient exhibits symptoms of such cancers.

In another aspect, the invention is directed to a method of making any of the embodiments described herein. In yet another aspect, the invention is directed to a method of using any of the embodiments described herein.

Other advantages, novel features, and objects of the invention will become apparent from the following detailed description of non-limiting embodiments of the invention when considered in conjunction with the accompanying drawings, which are schematic and which are not intended to be drawn to scale. In the figures, each identical or nearly identical component that is illustrated in various figures typically is represented by a single numeral. For purposes of clarity, not every component is labeled in every figure, nor is every component of each embodiment of the invention shown where illustration is not necessary to allow those of ordinary skill in the art to understand the invention. In cases where the present specification and a document incorporated by reference include conflicting disclosure, the present specification shall control.

### BRIEF DESCRIPTION OF THE DRAWINGS

Non-limiting embodiments of the present invention will be described by way of example with reference to the accompanying drawings in which:

FIG. 1 is a photocopy of a digital photo (original colors labeled in photocopy) of a colorimetric nanoparticle experiment;

FIG. 2 is a photocopy of a digital photo of a drug screening plate;

FIG. 3 is a bar graph illustrating certain compounds of the invention as used in an assay; and

FIG. 4 (sections A and B) is a photocopy of a digital photo of cells used in an angiogenesis assay.

### DETAILED DESCRIPTION OF THE INVENTION

One feature of the mechanism of angiogenesis involves cellular adhesion of vascular cells to extracellular matrices. Accordingly, one aspect of the present invention provides compositions able to function as angiogenesis inhibitors, for example, by preventing such adhesion and thus preventing the formation of structures such as that can initiate the production of new blood vessels. In one set of embodiments, these compositions may be selected with an assay that tests the ability of endostatin to bind to a portion of the protein, vitronectin, in the presence of the composition. In another set of embodiments, these compositions may be selected or validated with an assay that tests the ability of cells exposed to the composition, such as human umbilical vein endothelial cells (HUVEC), to participate in tubule formation characteristic of blood vessel formation.

In another set of embodiments, the invention is particularly directed to a patient population never before treated with the compositions useful according to certain methods of the invention, including patients who are not suffering from or indicating susceptibility to cell proliferation, cancer, or tumors, especially solid tumors. In other words, the treatment preferably is directed to patient populations that otherwise are free of symptoms that call for treatment with any of the compositions useful according to the invention.

One aspect of the invention includes compositions that are able to act as angiogenesis inhibitors. For example, the compositions have the ability to bind to alpha-V-beta-3 receptors, or the GRGDS motifs derived from vitronectin. Vitronectin is believed to be the biological target of the known angiogenesis inhibitor, endostatin, as further discussed in International patent application serial no. PCT/US01/46221, filed Nov. 15, 2001, published as WO 02/39999 on May 23, 2002, entitled Endostatin-Like Angiogenesis Inhibition, by Bamdad, et al, and U.S. patent application serial No. 10/003,681, filed Nov. 15, 2001, by Bamdad, et al, entitled “Endostatin-Like Angiogenesis Inhibition,”, each incorporated herein by reference. The compositions of the present invention are able to interrupt interactions between vitronectin and other native species required to promote angiogenesis.

International patent application serial number PCT/US01/12484, filed Apr. 12, 2001 by Bamdad et al., entitled “Treatment of Neurodegenerative Disease” (International patent publication WO 01/78709, published Oct. 25, 2001), International patent application serial number PCT/US00/01997, filed Jan. 25, 2000 by Bamdad et al., entitled “Rapid and Sensitive Detection of Aberrant Protein Aggregation in Neurodegenerative Diseases” (International patent publication WO 00/43791, published Jul. 27, 2000), and International patent application serial number PCT/US00/01504, filed Jan. 21, 2000 by Bamdad, et al., entitled “Interaction of Colloid-Immobilized Species with Species on Non-Colloidal Structures” (International patent publication WO 00/34783, published Jul. 27, 2000), all are incorporated herein by reference. Also incorporated herein by reference are the following: International patent application ser. no. PCT/US01/44782, filed Nov. 27, 2001 (publication WO 02/056022, publihed Jul. 18, 2002); U.S. patent application Ser. No. 09/631,818, filed Aug. 3, 2000, entitled “Rapid and Sensitive Detection of Protein Aggregation”; U.S. provisional patent application serial No. 60/213,763, filed Jun. 23, 2000, entitled “Detection of Binding Species with Colloidal and Non-Colloidal Structures”; U.S. provisional patent application No. 60/248,866 by Bamdad, et al., filed Nov. 15, 2000, entitled “Detection of Binding Species with Colloidal and Non-Colloidal Structures”; U.S. provisional patent application No. 60/248,865 by Bamdad, et al., filed Nov. 15, 2000, entitled “Endostatin-Like Angiogenesis Inhibition”; and U.S. provisional patent application serial No. 60/317,314, filed Sep. 5, 2001, entitled “Compositions and Methods of Treatment of Cancer,” by C. Bamdad et al. Also incorporated by reference is an application filed on even date herewith, entitled “Compositions and Methods of Treatment of Cancer,” by C. Bamdad, et al.

“Colloid,” as used herein, means nanoparticle, i.e. a very small, self-suspendable particles including inorganic, polymeric, and metal particles. Typically, colloid particles are of less than 250 nm cross section in any dimension, more typically less than 150 or 100 nm cross section in any dimension, and preferably 10-30 nm, and can be metal (for example, gold colloid particles), non-metal, crystalline or amorphous. As used herein this term includes the definition commonly used in the field of biochemistry.

The term “cancer,” as used herein, may include, but is not limited to, biliary tract cancer; bladder cancer; brain cancer including glioblastomas and medulloblastomas; breast cancer; cervical cancer; choriocarcinoma; colon cancer; endometrial cancer; esophageal cancer; gastric cancer; multiple myeloma; intraepithelial neoplasms including Bowen's disease and Paget's disease; liver cancer; lung cancer; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Kaposi's sarcoma, basocellular cancer, and squamous cell cancer; testicular cancer including germinal tumors such as seminoma, non-seminoma (teratomas, choriocarcinomas), stromal tumors and germ cell tumors; thyroid cancer including thyroid adenocarcinoma and medullar carcinoma; and renal cancer including adenocarcinoma and Wilms' tumor. Commonly encountered cancers include breast, prostate, lung, ovarian, colorectal, and brain cancer.

The term “cancer treatment” as described herein, may include, but is not limited to, chemotherapy, radiotherapy, adjuvant therapy, or any combination of the aforementioned methods. Aspects of treatment that may vary include, but are not limited to dosages, timing of administration or duration or therapy; and may or may not be combined with other treatments, which may also vary in dosage, timing, or duration. Another treatment for cancer is surgery, which can be utilized either alone or in combination with any of the aforementioned treatment methods. One of ordinary skill in the medical arts may determine an appropriate treatment for a patient.

A “subject” or a “patient,” as used herein, refers to any mammal (preferably, a human), and preferably a mammal that may be susceptible to tumorigenesis or cancer associated with the aberrant expression of MUC1. Examples include a human, a non-human primate, a cow, a horse, a pig, a sheep, a goat, a dog, a cat or a rodent such as a mouse, a rat, a hamster, or a guinea pig. Generally, or course, the invention is directed toward use with humans.

A “sample,” as used herein, is any cell, body tissue, or body fluid sample obtained from a subject. Preferred are body fluids include, for example, lymph, saliva, blood, urine, and the like. Samples of tissue and/or cells for use in the various methods described herein can be obtained through standard methods including, but not limited to, tissue biopsy, including punch biopsy and cell scraping, needle biopsy; or collection of blood or other bodily fluids by aspiration or other suitable methods.

Any additional definitions necessary for understanding the invention can be taken from International patent publication no. WO 02/056022, referenced above.

One aspect of the invention provides a pharmaceutical preparation comprising a composition comprising any of compositions shown below (numbered 1-31), optionally with a pharmaceutically acceptable carrier:

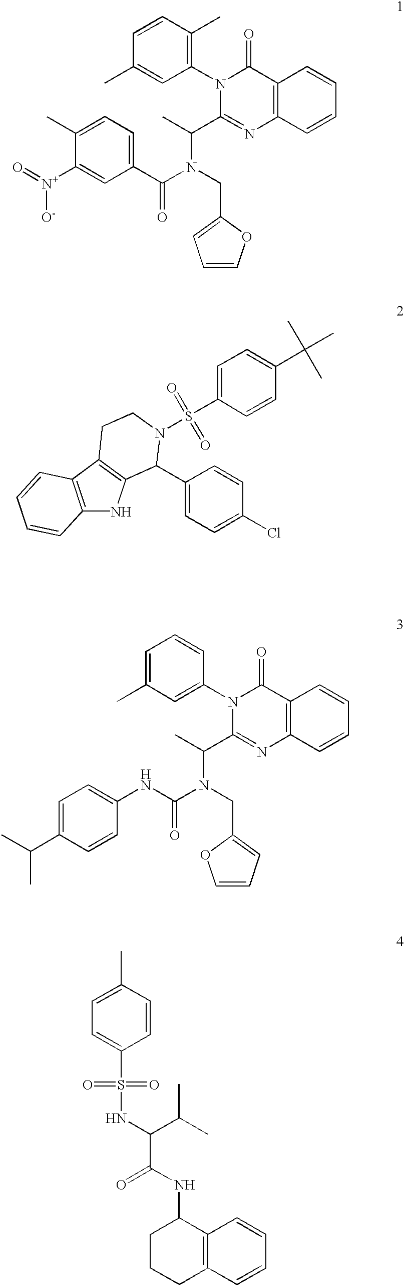

In one embodiment, the composition comprises homologs, analogs, derivatives, enantiomers and functionally equivalent compositions thereof of compositions 1-31. Another aspect of the present invention involves the utility of any of the above-mentioned compositions for the treatment of cancer and tumors, particularly solid tumors, by inhibition of angiogenesis associated with those tumors. In one embodiment, particularly preferred compositions are composition 3, 16, 18, 20 22 and 26.

In one aspect, the invention is defined, at least in part, by compositions having certain structures, as further described below. In these structures, the term “chemical bond” refers to any type of chemical bond, for example, a covalent bond, an ionic bond, a hydrogen bond, a van der Waals bond, a metal ligand bond, a dative bond, a hydrophobic interaction, or the like. Covalent bonds are preferred. In these structures, atoms able to form at least three covalent bonds include those atoms of the carbon family (e.g., carbon, silicon, or germanium), the nitrogen family (e.g., nitrogen, phosphorus, or arsenic), or the boron family (e.g., boron, aluminum, or gallium). In some embodiments, the atoms able to form at least three covalent bonds found within structures of the invention are carbon, nitrogen, silicon, and phosphorus, and in certain embodiments, the atoms are carbon and nitrogen.

The term “halogen,” or equivalently, “halogen atom,” is given its ordinary meaning as used in the field of chemistry. The halogens include fluorine, chlorine, bromine, iodine, and astatine. Preferably, the halogen atoms used in the present invention include one or more of fluorine, chlorine, bromine, or iodine. In certain embodiments of the invention, the halogen atoms found within the structure are fluorine, chlorine, and bromine; fluorine and chlorine; chlorine and bromine, or a single type of halogen atom.

As used herein, a “saturated” bond is given its ordinary meaning as used in the field of chemistry. A saturated moiety generally does not contain any double, triple, or higher order chemical bonds in its structure. The saturated moiety can contain any number or types of atoms (e.g., oxygen, carbon, nitrogen, hydrogen, or halogen atoms) in any configuration, so long as the moiety contains only single bonds between the atoms. For example, the saturated moiety may be an aliphatic structure or a cyclic structure. A saturated moiety may be connected to a parent structure at one or more points. Examples of saturated moieties include:

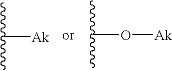

which each are connected to a parent structure at one point, or:

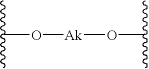

which is connected to a parent structure at more than one point (in this example, using ether linkages). In these structures, “Ak” refers to an alkyl group, as described below. As one example, the alkyl group in these structures may have one, two, three, or four carbon atoms, and may be straight-chained or branched, as long as no double or triple bonds are present. The alkyl group may also include only hydrogen atoms, or include halogen atoms as well.

Conversely, an “unsaturated” moiety is a moiety that contains at least one higher-order chemical bond within its structure, i.e., at least one double bond or triple bond between two atoms within its structure. The unsaturated moiety may contain, in some cases, more than one double and/or triple bond within its structure, for example, as in an alkadiene or an alkenyne.

As used herein, an “alkyl” is given its ordinary meaning as used in the field of organic chemistry. Alkyl or aliphatic groups typically contains any number of carbon atoms, for example, between 1 and 20 carbon atoms, between 1 and 15 carbon atoms, between 1 and 10 carbon atoms, or between 1 and 5 carbon atoms. In some embodiments, the alkyl group will contain at least 1 carbon atom, at least 2 carbon atoms, at least 3 carbon atoms, at least 4 carbon atoms, at least 5 carbon atoms, at least 6 carbon atoms, at least 7 carbon atoms, or at least 8 carbon atoms. Typically, an alkyl group is a non-cyclic structure. In certain embodiments, the alkyl group is a methyl group or an ethyl group.

The carbon atoms may be arranged in any configuration within the alkyl moiety, for example, as a straight chain (i.e., a n-alkyl such as methyl, ethyl, propyl, butyl, pentyl, hexyl, heptyl, octyl, nonyl, decyl, or undecyl) or a branched chain, for example, a t-butyl group, or an isoalkyl group such as isopropyl, isobutyl, ispentanyl, or isohexanyl. The alkyl moiety may contain none or any number of double or triple bonds within its structure, for example, as in an alkene, an alkyne, an alkadiene, an alkadiyne, an alkenyne, etc.

The alkyl group may contain any number of substituents. For example, the alkyl group may contain a halogen, an alkoxy (e.g., a methoxy, an ethoxy, a propoxy, an isopropoxy, a butoxy, a pentoxy, or the like), an amine (e.g., a primary, secondary, or tertiary amine, for example, an dimethylamine ethyl group), or a hydroxide as a substituent. As one example, if the alkyl group is a methyl group, then the methyl group may be substituted to form, for instance, a halogenated methyl group such as chloromethyl, bromomethyl, or iodomethyl. In some embodiments of the invention, more than one substituent may be present. For example, the alkyl group may have two or more halogen atoms (for example, two chlorine atoms, or a chlorine and a bromine atom), a halogen and an alkoxy group, or the like.

In some embodiments of the invention, the alkyl group may also contain one or more heteroatoms substituted within the alkyl group, such as a nitrogen atom (e.g., as in an amine such as a primary, secondary, or tertiary amine) or an oxygen atom (as in an ether moiety). However, in other embodiments of the invention, the main chain of the alkyl group is free of heteroatoms and includes carbon atoms. As used herein, the term “heteroatoms” refers to atoms that can replace carbon atoms within an alkyl group without affecting the connectivity of the alkyl group; these typically include oxygen and nitrogen atoms. Halogen atoms and hydrogen atoms are not considered to be heteroatoms; for example, a chlorine atom can replace a hydrogen atom within an alkyl group without affecting the connectivity of the alkyl group. As used herein, a “non-heteroatom alkyl group” is an alkyl group which does not contain any atoms at the carbon positions other than carbon. Some structures are defined as being free of non-terminal heteroatoms. As used herein, a “non-terminal” atom is an atom within a structure that is connected to at least two different atoms having a valency greater than 1 (e.g., the atom is connected to two non-hydrogen and non-halogen atoms). For example, the oxygen in —CH₂—OH and the nitrogen atom in —CH₂—NH₂ are not connected to two different atoms having a valency greater than 1, and thus are not non-terminal heteroatoms.

Similarly, a “cyclic” structure, as used herein, is given its ordinary definition in the field of organic chemistry, i.e., a structure that contains at least one ring of atoms, and may contain more than one ring of atoms. In other words, a cyclic structure has at least one chain of atoms that does not have a terminal end. The chain may have, for example, three, four, five, six, seven, or more atoms arranged to form a ring. The atoms within the chain may be carbon atoms, nitrogen atoms, oxygen atoms, silicon atoms, or any other atom that is able to bond to at least two different atoms.

In some embodiments of the invention, one or more substituents may be present on the cyclic structure. The substituents may be any substituent, as previously described in connection with alkyl moieties, for example, a halogen, an alkoxy, an amine, a hydroxide, or the like. In some embodiments, the substituents may also be alkyl groups, as previously described, for example, a methyl group, an ethyl group, a propyl group, and the like.

The cyclic structure may have one or more heteroatoms in some embodiments. For example, the cyclic structure may include a cyclohexane or a cyclopentane ring having one or more heteroatoms, such as:

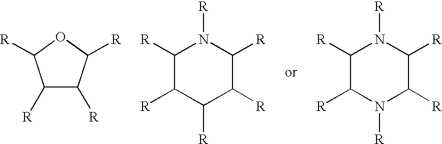

where the R's indicate the presence of additional atoms or substituents. The atoms substituted within the cyclohexane ring are able to form at least three covalent bonds, and, if able to form four covalent bonds, the fourth covalent bond may be attached to any atom.

The cyclic structure may be a saturated cyclic structure (such as a cyclohexyl or a cyclopentyl structure), or an unsaturated cyclic structure (such as a cyclohexenyl structure or an aromatic structure). Examples of aromatic structures, include, for instance, phenyl, naphthalenyl, anthacenyl, tolyl, pyridinyl, furanyl, pyrrolyl, and the like. A “nonaromatic cyclic structure” is a structure in which aromaticity of the cyclic structure is not present (for example, as in a saturated cyclic structure, a cycloalkenyl moiety, etc.)

In one set of embodiments, the aromatic structure includes a benzene ring. If substituents are present on the benzene ring (as previously discussed, for example, a halogen atom, a methyl group, a methoxy group, a trifluoromethyl group, etc.), they may be located in any position, i.e., in any ortho, meta, or para position, relative to the point of attachment of the benzene ring. If more than one substituent is present, then the substituents may be located at any available point within the benzene ring. For example, if there are two substituents, they may be located in the ortho and meta positions (i.e., in the 2,3 or 2,5 positions), the ortho and para positions, in the two ortho positions, in the two meta positions, or in the meta and para positions.

In one set of embodiments, the aromatic group is a nonsubstituted aromatic group, for example, a phenyl or a naphthalenyl group. In another set of embodiments, the aromatic structure is a halophenyl group or a dihalophenyl group, for example, 3-chloro-4-flurophenyl; o-, m-, or p-chlorophenyl; 2,4-difluorophenyl; or o-, m-, or p-bromophenyl. In another set of embodiments, the aromatic structure is a methylphenyl or a dimethyl phenyl group, for example, o-, m-, or p-methylphenyl; 2,3-dimethylphenyl; 2,4-dimethylphenyl; 2,5-dimethylphenyl. In another set of embodiments, the aromatic group is an alkylphenyl group, such as o-, m-, or p-methylphenyl; o-, m-, or p-ethylphenyl; 2-phenylethyl, or benzyl. In another set of embodiments, the aromatic structure is a halomethylphenyl group, such as 3-chloro-2-methylphenyl. In another set of embodiments, the aromatic structure is an alkoxyphenyl or a dialkoxyphenyl group, for example, o-, m-, or p-isopropoxyphenyl; o-, m-, or p-methoxyphenyl; o-, m-, or p-ethoxyphenyl; or 2,4-dimethoxyphenyl. In one set of embodiments, the aromatic group is fused with another ring of atoms. The second ring may be aromatic or nonaromatic. Examples include:

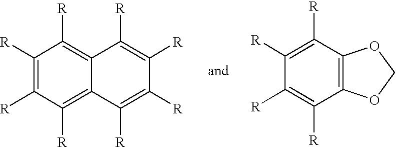

where the R's indicate the presence of additional atoms or substituents.

If the cyclic structure has more than one ring of atoms, the rings may be distributed in any manner within the moiety. For example, the two rings may not share a common atom, share only one common atom (e.g., as in a spiro-structure), or share more than one atom, as in a bicyclic structure or a propellane structure. If the two rings share at least one common chemical bond between two atoms, then the rings may be considered to be “fused.”

One example of a fused ring system is a structure:

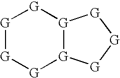

where a five member ring is fused to a six member ring in a bicyclic arrangement, and G represents an atoms each having at least three covalent bonds, as previously discussed. In some embodiments, one or both rings may be aromatic. As one example, a single nitrogen substitution onto the five-member ring, when both rings are aromatic, can result in an indole moiety, for example:

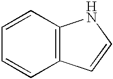

Additionally, other substituents or cyclic rings may be substituted onto the structure as well, for example, a cyclohexyl moiety.

If several rings are jointly fused to each other, then the rings may be considered to be “multifused.” One example of a multifused compound is an adamantane structure:

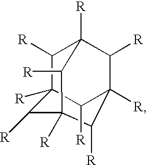

where the R's indicate the presence of additional atoms or substituents.

As used herein, when two cyclic groups are in a “branched configuration,” the two cyclic groups are on different branches of a common moiety. In other words, the two cyclic groups are not serially arranged relative to each other. That is, removal of either of the cyclic structures within the moiety does not automatically cause the other cyclic structure to be disconnected from the rest of the moiety. One example of this is illustrated by a diphenylmethyl moiety:

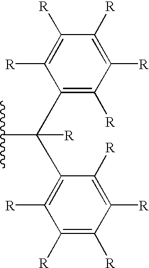

where the R's indicate the presence of additional atoms or substituents.

In one set of embodiments, the composition includes a substituted urea moiety. The substituted urea moiety includes at least one cyclic structure having at least seven members. In some cases, the cyclic structure may be a substituted cyclic structure, for example, the structure may include an azepane moiety or a cycloheptane structure, or the structure may include a cycloalkone moiety, that is, an oxygen atom that is double bonded to a member of the cyclic ring.

An “amino acid” is given its ordinary meaning as used in the field of biochemistry. An amino acid typically has a structure:

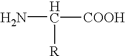

In this structure, R may be any suitable moiety. For example, R may be a hydrogen atom, a methyl group, or an isopropyl group. As used herein, the “natural amino acids” are the 20 amino acids commonly found in nature, i.e., alanine, arginine, asparagine, aspartic acid, cysteine, glutamine, glutamic acid, glycine, histidine, isoleucine, leucine, lysine, methionine, phenylalaine, proline, serine, threonine, tryptophan, tyrosine, and valine. Similarly, an unnatural amino acid is an amino acid, where the R group does not correspond to one of the natural amino acids.

In one set of embodiments, the composition comprises homologs, analogs, derivatives, enantiomers or functionally equivalent compositions thereof of the compositions of the present invention. Such homologs, analogs, derivatives, enantiomers or functionally equivalent compositions thereof of these compositions may be used for the treatment of cancer by inhibiting angiogenesis.

Homologs, analogs, derivatives, enantiomers and functionally equivalent compositions which are about as effective or more effective than the parent compound are intended for use in the method of the invention. Such compositions may also be screened by the assays described herein, for example, for increased potency and specificity towards treating or preventing cancer, cell proliferation, or angiogenesis, preferably with limited side effects. Synthesis of such compositions may be accomplished through typical chemical modification methods such as those routinely practiced in the art. As used herein, “functionally equivalent” generally refers to a composition that is capable of treatment of patients cancer, or of patients susceptible to cancer. It will be understood that one of ordinary skill in the art will be able to manipulate the conditions in a manner to prepare such homologs, analogs, derivatives, enantiomers and functionally equivalent compositions.

Another aspect of the invention provides a composition comprising any one of compositions of the present invention, and a homolog, analog, derivative, enantiomer or a functionally equivalent composition thereof capable of affecting angiogenesis.

Another aspect involves a method comprising providing any one of compositions of the present invention and performing a combinatorial synthesis on the composition, preferably to obtain homologs, analogs, derivatives, enantiomers and functionally equivalent compositions thereof of the composition. An assay may be performed with the homolog, analog, derivative, enantiomer or functionally equivalent composition to determine its effectiveness in functioning as an angiogenesis inhibitor. The combinatorial synthesis can involve subjecting a plurality of the compositions described herein to combinatorial synthesis.

Another aspect provides a method of administering any composition of the present invention to a subject. When administered, the compositions of the invention are applied in pharmaceutically acceptable amounts and as pharmaceutically acceptable compositions. Such preparations may routinely contain salts, buffering agents, preservatives, compatible carriers or other therapeutic ingredients. Examples of well-known carriers include glass, polystyrene, polypropylene, polyethylene, dextran, nylon, amylase, natural and modified cellulose, polyacrylamide, agarose and magnetite. The nature of the carrier can be either soluble or insoluble. Those skilled in the art will know of other suitable carriers, or will be able to ascertain such, using only routine experimentation.

In some cases, the present invention includes the step of bringing a composition of the invention into association or contact with a suitable carrier, which may constitute one or more accessory ingredients. The final compositions may be prepared by any suitable technique, for example, by uniformly and intimately bringing the composition into association with a liquid carrier, a finely divided solid carrier or both, optionally with one or more formulation ingredients such as buffers, emulsifiers, diluents, excipients, drying agents, antioxidants, preservatives, binding agents, chelating agents, or stabilizers and then, if necessary, shaping the product.

In some embodiments, the compositions of the present invention may be present as a pharmaceutically acceptable salt. The term “pharmaceutically acceptable salts” includes salts of the composition, prepared, for example, with acids or bases, depending on the particular substituents found within the composition and the treatment modality desired. Pharmaceutically acceptable salts can be prepared as alkaline metal salts, such as lithium, sodium, or potassium salts; or as alkaline earth salts, such as beryllium, magnesium or calcium salts. Examples of suitable bases that may be used to form salts include ammonium, or mineral bases such as sodium hydroxide, lithium hydroxide, potassium hydroxide, calcium hydroxide, magnesium hydroxide, and the like. Examples of suitable acids that may be used to form salts include inorganic or mineral acids such as hydrochloric, hydrobromic, hydroiodic, hydrofluoric, nitric, carbonic, monohydrogencarbonic, phosphoric, monohydrogenphosphoric, dihydrogenphosphoric, sulfuric, monohydrogensulfuric, phosphorous acids and the like. Other suitable acids include organic acids, for example, acetic, propionic, isobutyric, maleic, malonic, benzoic, succinic, suberic, fumaric, mandelic, phthalic, benzenesulfonic, p-tolylsulfonic, citric, tartaric, methanesulfonic, glucuronic, galactunoric, salicylic, formic, naphthalene-2-sulfonic, and the like. Still other suitable acids include amino acids such as arginate, aspartate, glutamate, and the like.

In general, pharmaceutically acceptable carriers for are well-known to those of ordinary skill in the art. As used herein, a “pharmaceutically acceptable carrier” refers to a non-toxic material that does not significantly interfere with the effectiveness of the biological activity of the active ingredient or ingredients. Pharmaceutically acceptable carriers include, for example, diluents, emulsifiers, fillers, salts, buffers, excipients, drying agents, antioxidants, preservatives, binding agents, bulking agents, chelating agents, stabilizers, solubilizers, and other materials well-known in the art. Examples of suitable formulation ingredients include diluents such as calcium carbonate, sodium carbonate, lactose, kaolin, calcium phosphate, or sodium phosphate; granulating and disintegrating agents such as corn starch or algenic acid; binding agents such as starch, gelatin or acacia; lubricating agents such as magnesium stearate, stearic acid, or talc; time-delay materials such as glycerol monostearate or glycerol distearate; suspending agents such as sodium carboxymethylcellulose, methylcellulose, hydroxypropylmethylcellulose, sodiumalginate, polyvinylpyrrolideone; dispersing or wetting agents such as lecithin or other naturally-occurring phosphatides; or thickening agents such as cetyl alcohol or beeswax. The compositions of the invention may be formulated into preparations in solid, semi-solid, liquid or gaseous forms such as tablets, capsules, elixrs, powders, granules, ointments, solutions, depositories, inhalants or injectables. The compositions of the present invention may be delivered by any suitable delivery method, for example, oral, parenteral or surgical administration. The invention also embraces locally administering the compositions of the invention, for example, as implants

Preparations include sterile aqueous or nonaqueous solutions, suspensions and emulsions. Examples of nonaqueous solvents are propylene glycol, polyethylene glycol, vegetable oil such as olive oil, an injectable organic esters such as ethyloliate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers, (such as those based on Ringer's dextrose), and the like. Preservatives and other additives may also be present such as, for example, antimicrobials, antioxidants, chelating agents and inert gases and the like. Those of skill in the art can readily determine the various parameters for preparing these pharmaceutical compositions without resort to undue experimentation.

Compositions of the invention may be administered singly or in combination with other compositions of the invention or other compositions. For example, in one embodiment, compositions of the invention are administered in combination with agents that inhibit angiogenesis, for example, by targeting or blocking cell surface receptors, such as the alpha-V-beta-3 cell surface receptor.

According to the methods of the invention, the compositions of the invention can be administered by injection by gradual infusion over time or by any other medically acceptable mode. Any medically acceptable method may be used to administer the composition to the patient. The particular mode selected will depend of course, upon factors such as the particular drug selected, the severity of the state of the subject being treated, or the dosage required for therapeutic efficacy. The methods of this invention, generally speaking, may be practiced using any mode of administration that is medically acceptable, meaning any mode that produces effective levels of the active composition without causing clinically unacceptable adverse effects.

The administration may be localized (i.e., to a particular region, physiological system, tissue, organ, or cell type) or systemic, depending on the condition to be treated. For example, the composition may be administered through parental injection, implantation, orally, vaginally, rectally, buccally, pulmonary, topically, nasally, transdermally, surgical administration, or any other method of administration where access to the target by the composition is achieved. Examples of parental modalities that can be used with the invention include intravenous, intradermal, subcutaneous, intracavity, intramuscular, intraperitoneal, epidural, or intrathecal. Examples of implantation modalities include any implantable or injectable drug delivery system. Oral administration may be preferred for some treatments because of the convenience to the patient as well as the dosing schedule. Compositions suitable for oral administration may be presented as discrete units such as capsules, pills, cachettes, tables, or lozenges, each containing a predetermined amount of the active compound. Other oral compositions include suspensions in aqueous or non-aqueous liquids such as a syrup, an elixir, or an emulsion.

The compositions of the present invention may be given in dosages, generally, at the maximum amount while avoiding or minimizing any potentially detrimental side effects. The compositions can be administered in effective amounts, alone or in a cocktail with other compounds, for example, other compounds that can be used to treat cancer or tumorigenesis. An effective amount is generally an amount sufficient to inhibit angiogenesis of tumors within the subject.

One of skill in the art can determine what an effective amount of the composition is by screening the ability of the composition using any of the assays described herein. The effective amounts will depend, of course, on factors such as the severity of the condition being treated; individual patient parameters including age, physical condition, size and weight; concurrent treatments; the frequency of treatment; or the mode of administration. These factors are well known to those of ordinary skill in the art and can be addressed with no more than routine experimentation. It is generally preferred that a maximum dose be used, that is, the highest safe dose according to sound medical judgment.

Dosages may be estimated based on the results of experimental models, optionally in combination with the results of assays of the present invention. Generally, daily oral prophylactic doses of active compounds will be from about 0.01 mg/kg per day to 2000 mg/kg per day. Oral doses in the range of 10 to 500 mg/kg, in one or several administrations per day, may yield suitable results. In the event that the response of a particular subject is insufficient at such doses, even higher doses (or effective higher doses by a different, more localized delivery route) may be employed to the extent that patient tolerance permits. Multiple doses per day are also contemplated in some cases to achieve appropriate systemic levels of the composition.

In administering the compositions of the invention to subjects, dosing amounts, dosing schedules, routes of administration and the like may be selected so as to affect other known activities of these compositions. For example, amounts, dosing schedules and routes of administration may be selected as described herein, whereby therapeutically effective levels for angiogenesis inhibition are provided, yet therapeutically effective levels for alternative treatments are not provided.

Other delivery systems suitable for use with the present invention include time-release, delayed release, sustained release, or controlled release delivery systems. Such systems may avoid repeated administrations of the active compounds of the invention in many cases, increasing convenience to the subject and the physician. Many types of release delivery systems are available and known to those of ordinary skill in the art. They include, for example, polymer based systems such as polylactic and/or polyglycolic acid, polyanhydrides, and polycaprolactone; nonpolymer systems that are lipid-based including sterols such as cholesterol, cholesterol esters, and fatty acids or neutral fats such as mono-, di- and triglycerides; hydrogel release systems; silastic systems; peptide based systems; wax coatings; compressed tablets using conventional binders and excipients; or partially fused implants. Specific examples include, but are not limited to, erosional systems in which the composition is contained in a form within a matrix, or diffusional systems in which an active component controls the release rate. The formulation may be as, for example, microspheres, hydrogels, polymeric reservoirs, cholesterol matrices, or polymeric systems. In some embodiments, the system may allow sustained or controlled release of the active compound to occur, for example, through control of the diffusion or erosion/degradation rate of the formulation. In addition, a pump-based hardware delivery system may be used in some embodiment of the invention.

Use of a long-term release implant may be particularly suitable in some cases. “Long-term release,” as used herein, means that the implant is constructed and arranged to deliver therapeutic levels of the composition for at least 30 or 45 days, and preferably at least 60 or 90 days, or even longer in some cases. Long-term release implants are well known to those of ordinary skill in the art, and include some of the release systems described above.

The present invention also provides any of the above-mentioned compositions useful for the treatment of solid tumors packaged in kits, optionally including instructions for use of the composition for the treatment of cancer. That is, the kit can include a description of use of the composition for participation in any biological or chemical mechanism disclosed herein associated with cancer or tumorigenesis. The kit can include a description of use of the compositions as discussed herein. The kit also can include instructions for use of a combination of two or more compositions of the invention. Instructions also may be provided for administering the drug by any suitable technique, such as orally, intravenously, directly into the cerebrospinal fluid via a spinal drip, pump or implantable delivery device, or via another known route of drug delivery. The invention also involves promotion of the treatment of solid tumors according to any of the techniques and compositions and composition combinations described herein.

The compositions of the invention, in some embodiments, may be promoted for treatment of abnormal cell proliferation, cancers, or tumors, particularly solid tumors, or includes instructions for treatment of accompany cell proliferation, cancers, or tumors, particularly solid tumors, as mentioned above. In another aspect, the invention provides a method involving promoting the prevention or treatment of cancer via administration of any one of the compositions of the present invention, and homologs, analogs, derivatives, enantiomers and functionally equivalent compositions thereof in which the composition is able to function as an angiogenesis inhibitor. The compositions of the invention may be promoted to affect angiogenesis. The invention may also include instructions for the treatment of cancers by inhibiting angiogenesis. As used herein, “promoted” includes all methods of doing business including methods of education, hospital and other clinical instruction, pharmaceutical industry activity including pharmaceutical sales, and any advertising or other promotional activity including written, oral and electronic communication of any form, associated with compositions of the invention in connection with treatment of cell proliferation, cancers or tumors. “Instructions” can define a component of promotion, and typically involve written instructions on or associated with packaging of compositions of the invention. Instructions also can include any oral or electronic instructions provided in any manner. The “kit” typically defines a package including any one or a combination of the compositions of the invention, or homologs, analogs, derivatives, enantiomers and functionally equivalent compositions thereof, and the instructions, but can also include the composition of the invention and instructions of any form that are provided in connection with the composition in a manner such that a clinical professional will clearly recognize that the instructions are to be associated with the specific composition. The kit can include a description of use of the composition for participation in any angiogenesis mechanism that is associated with cancer or tumorigenesis. These and other embodiments of the invention can also involve promotion of the treatment of cancer or tumorigenesis according to any of the techniques and compositions and combinations of compositions described herein.

The kits described herein may also contain one or more containers, which can contain compounds such as the species, signaling entities, biomolecules and/or particles as described. The kits also may contain instructions for mixing, diluting, and/or administrating the compounds. The kits also can include other containers with one or more solvents, surfactants, preservative and/or diluents (e.g., normal saline (0.9% NaCl), or 5% dextrose) as well as containers for mixing, diluting or administering the components to the sample or to the patient in need of such treatment.

The compositions of the kit may be provided as any suitable form, for example, as liquid solutions or as dried powders. When the composition provided is a dry powder, the powder may be reconstituted by the addition of a suitable solvent, which may also be provided. In embodiments where liquid forms of the composition are sued, the liquid form may be concentrated or ready to use. The solvent will depend on the compound and the mode of use or administration. Suitable solvents for drug compositions are well known and are available in the literature. The solvent will depend on the compound and the mode of use or administration.

The kit, in one set of embodiments, may comprise a carrier means being compartmentalized to receive in close confinement one or more container means such as vials, tubes, and the like, each of the container means comprising one of the separate elements to be used in the method. For example, one of the container means may comprise a positive control in the assay. Additionally, the kit may include containers for other components, for example, buffers useful in the assay.

The function and advantage of these and other embodiments of the present invention will be more fully understood from the examples below. The following examples are intended to illustrate the benefits of the present invention, but do not exemplify the full scope of the invention.

### EXAMPLE 1

In this example, the angiogenesis inhibitor, endostatin, is specifically bound to a His-tagged GRGDS motif peptide (HHHHHHSSSSGSSSSGSSSSGGRGDSGRGDS) derived from vitronectin, whereas angiostatin is not bound.

200 microliters NTA-Ni²⁺ agarose were washed twice with 100 microliters of ddH₂O, then with “Buffer A,” containing 50 mM NaH₂PO₄, 300 mM NaCl, and 10 mM imidazole at pH 8.0.

A synthetic peptide, (HHHHHHSSSSGSSSSGSSSSGGRGDSGRGDS, derived from vitronectin, hereafter referred to as “GRGDS peptide”), was dissolved in dimethyl sulfoxide, then diluted in phosphate buffer to a final concentration of 1 mM. 100 microliters of this peptide solution were incubated with the NTA-Ni²⁺ resin for 20 minutes at room temperature, allowing binding of the histidine-tagged peptide to the NTA-Ni²⁺ resin to occur. The resin was then pelleted and the supernatant removed. The resin was then washed in Buffer A. The peptide-bound resin was then divided into two aliquots.

One aliquot was mixed with 100 microliters human recombinant endostatin (0.1 mg/mL in 10 mM sodium phosphate buffer, 100 mM sodium chloride, pH 7.4, diluted from a stock solution of endostatin). A second aliquot was mixed with 100 microliters of human angiostatin (0.1 mg/mL in 10 mM sodium phosphate buffer, 100 mM sodium chloride, pH 7.4). The beads and angiogenesis inhibitors were incubated on ice for 15-20 minutes, allowing binding to the bead-immobilized peptide to occur. The resin was then pelleted. The supernatants were removed and reserved for analysis by SDS-PAGE (flow through). The beads were then washed twice with 10 mM sodium phosphate buffer solution. The histidine-tagged peptides and any immobilized drug were eluted by the addition of 4 aliquots of an imidazole (250 mM) wash.

Analysis of the eluate and flow through by SDS-PAGE was then performed. This analysis showed that endostatin co-eluted with the GRGDS motif peptide, but angiostatin and other control proteins did not.

### EXAMPLE 2

This example illustrates that vitronectin inhibits binding of endostatin to the GRGDS peptide.

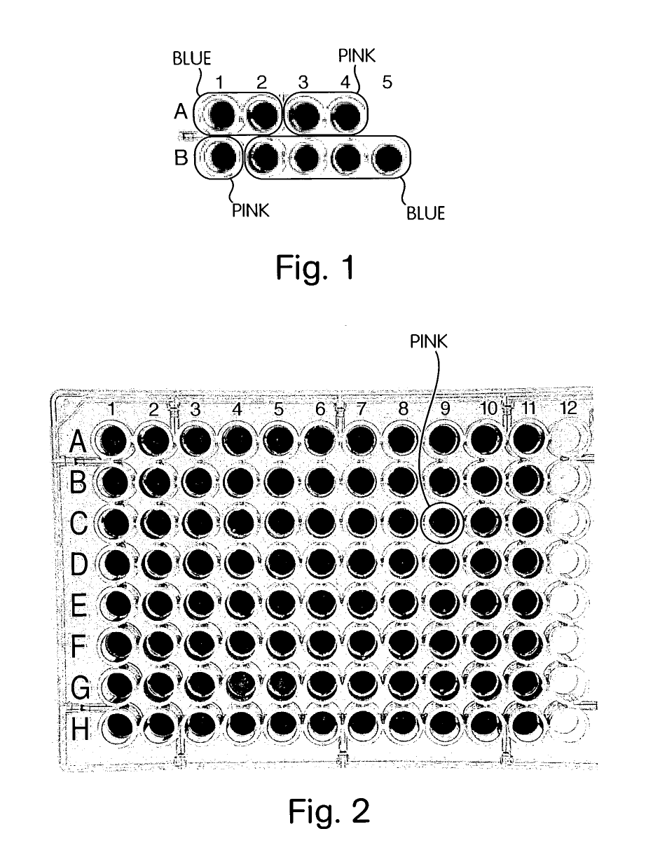

40 μM NTA gold colloids were prepared which presented the His-tagged GRGDS peptide. These colloids were mixed with endostatin (0.1 mg/mL) and turned blue, indicating binding of endostatin to the GRGDS peptide (A1 and A2 in FIG. 1). Control colloids presenting an irrelevant FLR-peptide (GTINVHDVETQFNQYKTEAASPYNLTISDVSVSDVPFPFSAQSGAHHHHHH) remained pink (wells A3 and A4 in FIG. 1). At the highest concentration of vitronectin (0.1 mg/ml), the endostatin-GRGDS interaction was disrupted, and the well remains pink (well B1). At lower concentrations of vitronectin, the endostatin-GRGDS interaction was not affected and the wells turn blue (wells B2 to B5).

FIG. 1 is a photocopy of a digital photo (original in color, original colors labeled) of a colorimetric nanoparticle experiment showing that the GRGDS-containing peptide interacted with dimeric endostatin (wells A1 and A2), and that this interaction was competitively inhibited by the addition of full-length vitronectin (well B1).

### EXAMPLE 3

This example illustrates a drug screen for angiogenesis inhibitors that functions by blocking the interaction between a portion of vitronectin and native proteins that may otherwise promote angiogenesis.

40 μM NTA colloids presenting a histidine-tagged peptide containing a tandem repeat GRGDS motif were prepared by incubating 2.1 mL colloids with 210 microliters 100 micromolar histidine-GRGDS for ten minutes pelleting the colloids to remove excess unbound peptide, and resuspending the colloids in 10 mM sodium phosphate buffer (pH 7.4).

Negative control colloids were prepared by substituting an irrelevant His-tagged FLR peptide (GTINVHDVETQFNQYKTEAASPYNLTISDVSVSDVPFPFSAQSGAHHHHHH). 25 microliters of GRGDS-colloids (or random peptide-colloid for negative controls) were added to each well of a 96-well plate, along with 65 microliters of sodium phosphate buffer solution per well. Dimethyl sulfoxide was added in place of a drug to the positive and negative controls. 5 microliters of 0.1 mg/ml endostatin were added to each well. The plate was then incubated in room temperature and observed for color change.

After about 20 minutes, the positive controls changed color from pink to blue as the endostatin bound to the GRGDS peptide. However, the negative control wells remained pink, since endostatin did not bind to the random peptide. A color change from pink to blue in the wells containing drug candidates indicates that the drug did not effect binding of endostatin to GRGDS. A lack of color change from pink to blue (i.e., the well remains pink over time) indicates that the drug candidate had bound to either the GRGDS peptide or endostatin, disrupting the binding interaction between endostatin and the GRGDS peptide. Drugs identified in this manner are useful as angiogenesis inhibitors.

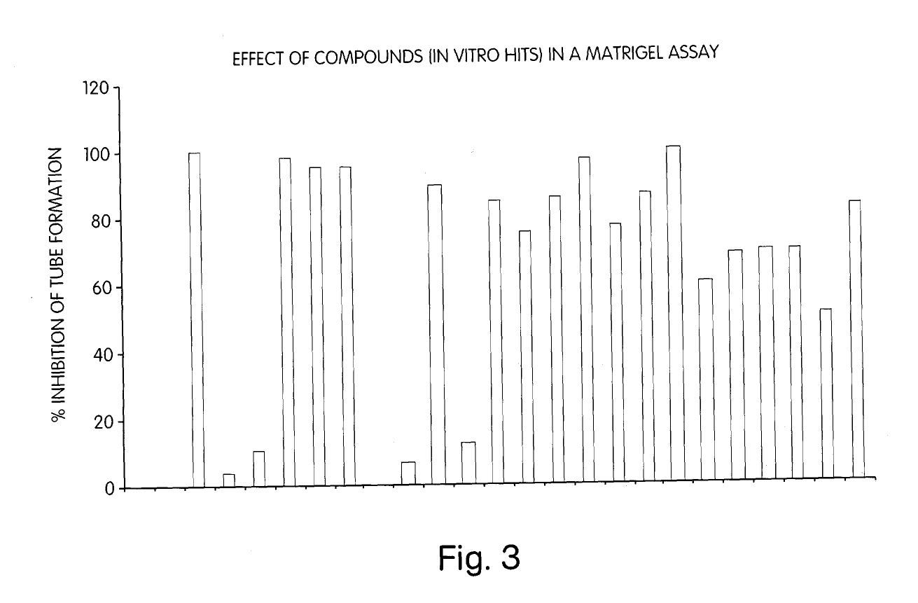

FIG. 2 is a photocopy of a digital photo of a drug screening plate in which drug candidates were separately tested in wells of a multi-well plate for their ability to interrupt the endostatin-GRGDS-containing peptide interaction. For example, the pink color of well C9 indicates that it contains a drug that mimics endostatin.

### EXAMPLE 4

This example illustrates an in vitro assay for testing angiogenesis inhibitors. In this example, a functional assay demonstrates that the compounds selected in the high throughput assay, described above in Example 3, effectively inhibit the process of tubule formation when tested as follows in an angiogenesis-inhibition assay. In this example, certain compositions were screened for the capability to prevent MATRIGEL®-induced capillary tube formation, which is indicative of the formation of blood vessels. This assay was performed generally following a method described by the manufacturer of MATRIGEL® (Becton Dickinson, San Jose, Calif.), a basement membrane matrix extracted from Engelbreth-Holm-Swarm mouse sarcoma.

The membrane matrix was diluted to 4 mg/mL with cold phosphate-buffered saline (PBS) and added to 24-well plates for a total volume of 200 microliters in each well. The plates were allowed to stand at 37° C. for 30 min. to form a gel layer. After gel formation, human umbilical vein endothelial cells (HUVECs) (about 2×10⁵ cells in a medium specific for growing endothelial cells, candidate compositions to be tested or a control (e.g. dimethyl sulfoxide) were applied to each well. The plates were incubated at 37° C. for 24 h with 5% CO₂. After incubation, the cells were washed and fixed in 2% glutaldehyde for 10 min.

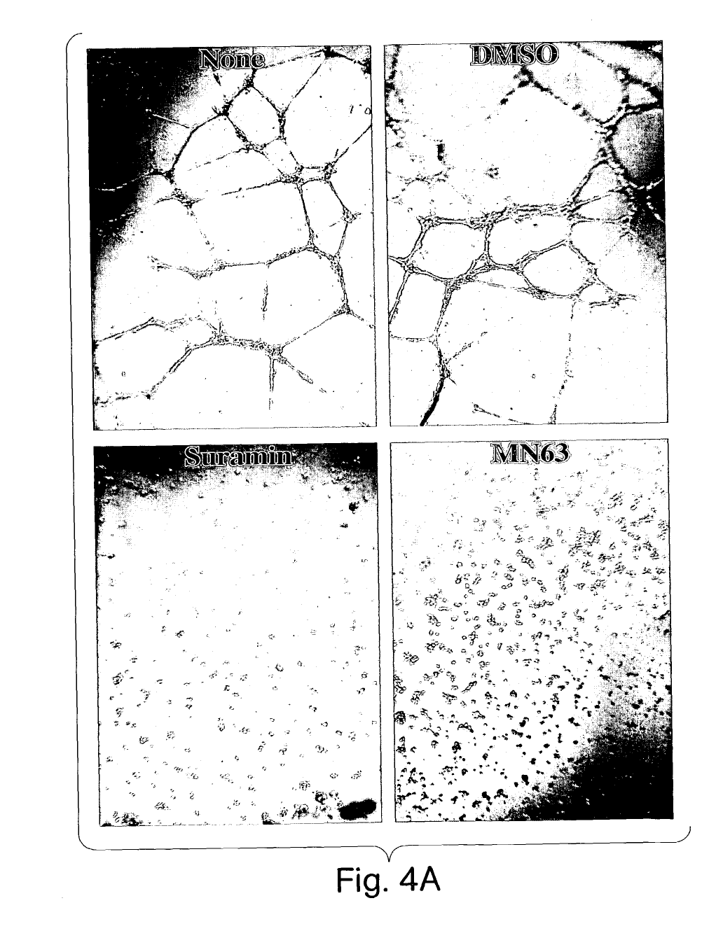

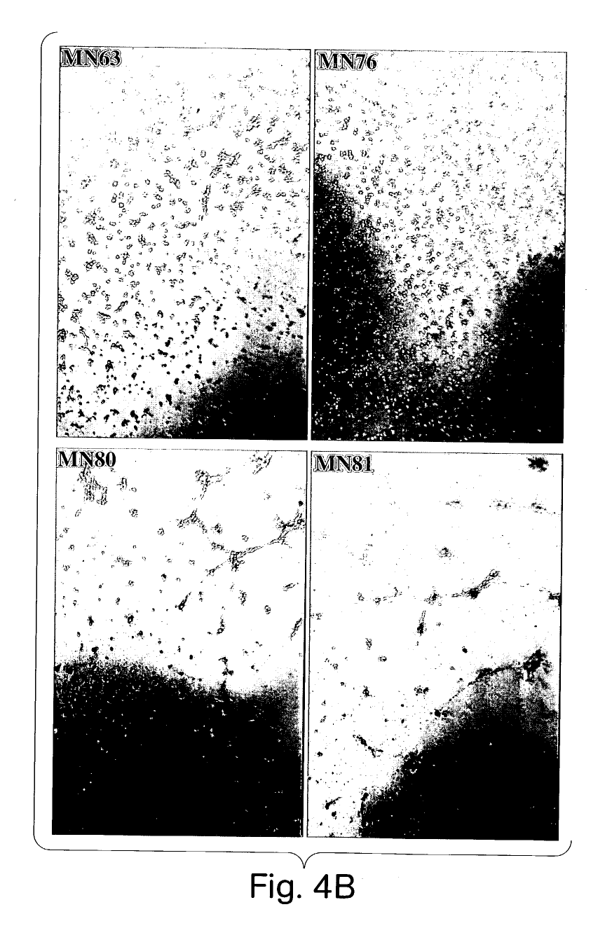

The cells were subjected to inverted contrast-phase microscopy and photographed. Successful candidate compositions resulted in cells that did not show capillary tube formation. FIG. 3 is a bar graph that reflects the ability of several compositions of the invention to inhibit tubule formation in this assay. FIG. 4 is photocopy of a set of photographs that demonstrate the activity of selected compositions of the invention compared to controls and known angiogenesis inhibitors. Drugs which prevented the formation of these tubule structures were scored as angiogenesis inhibitors.

While several embodiments of the invention have been described and illustrated herein, those of ordinary skill in the art will readily envision a variety of other means and structures for performing the functions and/or obtaining the results or advantages described herein, and each of such variations or modifications is deemed to be within the scope of the present invention. More generally, those skilled in the art would readily appreciate that all parameters, dimensions, materials, and configurations described herein are meant to be exemplary and that actual parameters, dimensions, materials, and configurations will depend upon specific applications for which the teachings of the present invention are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described. The present invention is directed to each individual feature, system, material and/or method described herein. In addition, any combination of two or more such features, systems, materials and/or methods, if such features, systems, materials and/or methods are not mutually inconsistent, is included within the scope of the present invention.

In the claims (as well as in the specification above), all transitional phrases such as “comprising,” “including,” “carrying,” “having,” “containing,” “involving,” and the like are to be understood to be open-ended, i.e. to mean including but not limited to. Only the transitional phrases “consisting of” and “consisting essentially of” shall be closed or semi-closed transitional phrases, respectively, as set forth in the United States Patent Office Manual of Patent Examining Procedures, section 2111.03.

What is claimed is:

14. A composition, comprising a structure: 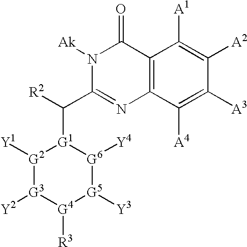
wherein A¹, A², A³, and A⁴ are each independently selected from the group consisting of H and a halogen,
Y¹, Y², Y³, R² and R³ each independently comprise an atom,
G¹, G², G³, G⁴, G⁵, and G⁶ each independently comprise an atom able to form at least three covalent bonds, and
Ak comprises an alkyl.

15. A composition, comprising a structure: 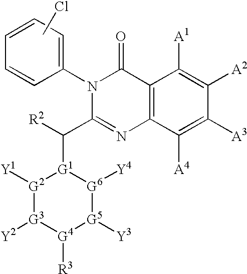
wherein A¹, A², A³, and A⁴ are each independently selected from the group consisting of H and a halogen,
Y¹, Y², Y³, Y⁴, R² and R³ each independently comprise an atom, and
G¹, G², G³, G⁴, G⁵, and G⁶ each independently comprise an atom able to form at least three covalent bonds.

16. A composition, comprising a structure: 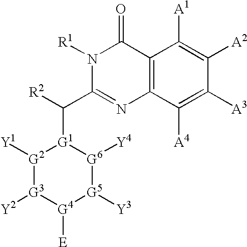
wherein A¹, A², A³, and A⁴ are each independently selected from the group consisting of H and a halogen,
Y¹, Y², Y³, Y⁴, R¹ and R² each independently comprise an atom,
G¹, G², G³, G⁴, G⁵, and G⁶ each independently comprise an atom able to form at least three covalent bonds, and
E comprises a sulfur atom.

17. A composition, comprising a structure: 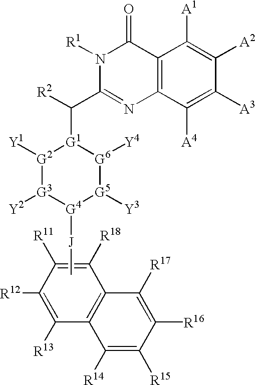
wherein A¹, A², A³, and A⁴ are each independently selected from the group consisting of H and a halogen,
Y¹, Y², Y³, Y⁴, R¹, R², R¹¹, R¹², R¹³, R¹⁴, R¹⁵, R¹⁶, R¹⁷, and R¹⁸ each independently comprise an atom,
G¹, G², G³, G⁴, G⁵, and G⁶ each independently comprise an atom able to form at least three covalent bonds, and
J comprises a chemical bond or an atom.

18. A composition, comprising a structure: 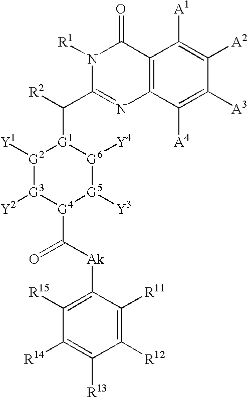
wherein A¹, A², A³, and A⁴ are each independently selected from the group consisting of H and a halogen,
Y¹, Y², Y³, Y⁴, R¹, R², R¹¹, R¹², R¹³, R¹⁴, and R¹⁵ each independently comprise an atom, and
G¹, G², G³, G⁴, G⁵, and G⁶ each independently comprise an atom able to form at least three covalent bonds.

19. A composition, comprising a structure: 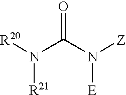
wherein R²⁰ and R²¹ each independently comprise an atom,
E comprises at least 2 cyclic groups, and
Z comprises at least two fused cyclic structures;
in combination with a pharmaceutically acceptable carrier.

20. A composition, comprising a structure: 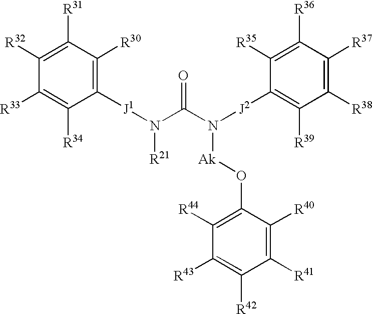
wherein Ak comprises a non-heteroatom alkyl group,
R²¹, R³⁰, R³¹, R³², R³³, R³⁴, R³⁵, R³⁶, R³⁷, R³⁸, R³⁹, R⁴⁰, R⁴¹, R⁴², R⁴³, and R⁴⁴ each independently comprise an atom, and
J¹ and J² each independently comprise a chemical bond or an atom;
in combination with a pharmaceutically acceptable carrier.

21. A composition, comprising a structure: 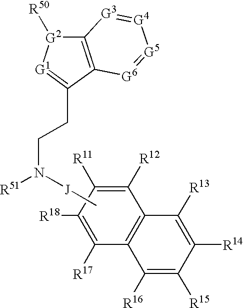
wherein R¹¹, R¹², R¹³, R¹⁴, R¹⁵, R¹⁶, R¹⁷, R¹⁸, R⁵⁰, and R⁵¹ each independently comprise an atom,
G¹, G², G³, G⁴, and G⁵ each independently comprise an atom able to form at least three covalent bonds, and
J comprises a chemical bond or an atom;
in combination with a pharmaceutically acceptable carrier.

22. A composition, comprising a structure: 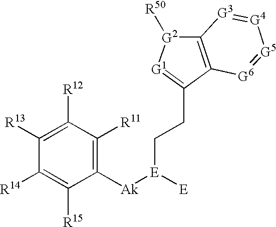
wherein R¹¹, R¹², R¹³, R¹⁴, R¹⁵, and R⁵⁰ each independently comprise an atom,
G¹, G², G³, G⁴, and G⁵ each independently comprise an atom able to form at least three covalent bonds,
Ak comprises an alkyl, and
E comprises a sulfur atom;
in combination with a pharmaceutically acceptable carrier.

23. A composition, comprising a structure: 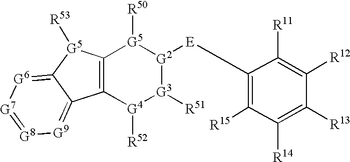
wherein R¹¹, R¹², R¹³, R¹⁴, R¹⁵, R⁵⁰, R⁵¹, R⁵², and R⁵³ each independently comprise an atom,
G¹, G², G³, G⁴, G⁵, G⁶, G⁷, G⁸, and G⁹ each independently comprise an atom able to form at least three covalent bonds, and
E comprises a sulfur atom;
in combination with a pharmaceutically acceptable carrier.

24. A method, comprising:
treating a human patient susceptible to or exhibiting a solid tumor, by administering to the patient a therapeutically effective amount of a composition that inhibits the tumor by inhibiting angiogenesis, comprising: 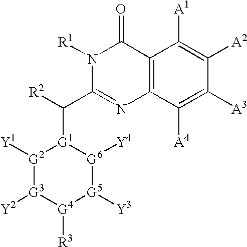
wherein A¹, A², A³, and A⁴ are each independently selected from the group consisting of H and a halogen,
Y¹, Y², Y³, Y⁴, R¹, R² and R³ each independently comprise an atom,
G¹, G², G³, G⁴, G⁵, and G⁶ each independently comprise an atom able to form at least three covalent bonds, and
the patient is not otherwise indicated for treatment with the composition.

25. The method of claim 24, wherein the composition targets vitronectin.

26. The method of claim 24, wherein the composition targets the alpha-V-beta-3 receptor.

27. The method of claim 24, comprising treating the patient with the composition as recited in claim 24 in combination with at least one other active agent.

28. The method of claim 27, wherein the at least one other active agent is selected to inhibits the tumor by inhibiting angiogenesis.
